(19) United States
(12) Patent Application Publication
(10) Pub. No.: US 2023/0390428 A1
(43) Pub. Date: Dec. 7, 2023

(54) ULTRAVIOLET LIGHT SOURCE FOR USE IN AN ENVIRONMENT FOR HUMAN OCCUPATION INCLUDING HARDWARE SAFETY INTERLOCKS

(71) Applicant: CURRENT LIGHTING SOLUTIONS, LLC, East Cleveland, OH (US)

(72) Inventor: Stephen P. Glaudel, East Cleveland, OH (US)

(73) Assignee: CURRENT LIGHTING SOLUTIONS, LLC, East Cleveland, OH (US)

(21) Appl. No.: 18/031,929

(22) PCT Filed: Oct. 7, 2021
(86) PCT No.: PCT/US2021/053922

§ 371 Date: Apr. 14, 2023

Related U.S. Application Data
(60) Provisional application No. 63/092,143, filed on Oct. 15, 2020.

(51) Int. Cl. A61L 2/00; A61L 9/20; A61L 2/10; A61L 2/24

(52) U.S. Cl. CPC A61L 2/0047; A61L 9/20; A61L 2/10; A61L 2/24; A61L 2202/14

(57) ABSTRACT

A disinfection light source comprises one or more disinfection light emitters and electronics conducting electric power to the disinfection light emitters to drive them to emit light. The electronics include a safety interlock circuit that includes: an ammeter measuring electric current flowing through the one or more disinfection light emitters; an analysis circuit analyzing the electric current measured by the ammeter; a timer circuit configured to run while the analysis circuit detects a high irradiation condition; and a switch to interrupt the conduction of the electric power to the one or more disinfection light emitters in response to the timer circuit running and reaching a delay interval of the timer circuit. The analysis circuit may comprise a comparator comparing the electric current measured by the ammeter with an electrical reference value, and the timer circuit then runs while the comparator indicates the electric current exceeds the electrical reference value.

This application claims the benefit of U.S. Provisional Application No. 63/092,143 filed Oct. 15, 2020 titled “ULTRAVIOLET LIGHT SOURCE FOR USE IN AN ENVIRONMENT FOR HUMAN OCCUPATION INCLUDING HARDWARE SAFETY INTERLOCKS”. U.S. Provisional Application No. 63/092,143 filed Oct. 15, 2020 titled “ULTRAVIOLET LIGHT SOURCE FOR USE IN AN ENVIRONMENT FOR HUMAN OCCUPATION INCLUDING HARDWARE SAFETY INTERLOCKS” is incorporated herein by reference in its entirety.

### BACKGROUND

The following relates to the disinfection arts, pathogen control arts, bacterial pathogen control arts, lighting arts, and the like.

Clynne et al., U.S. Pat. No. 9,937,274 B2 issued Apr. 10, 2018 and Clynne et al., U.S. Pat. No. 9,981,052 B2 (which is a continuation of U.S. Pat. No. 9,937,274) provide, in some illustrative examples, disinfection systems that includes a light source configured to generate ultraviolet light toward one or more surfaces or materials to inactivate one or more pathogens on the one or more surfaces or materials.

U.S. Pub. No. 2016/0271281 A1 is the published application corresponding to U.S. Pat. No. 9,937,274. U.S. Pub. No. 2016/0271281 A1 is incorporated herein by reference in its entirety to provide general information on disinfection systems for occupied spaces that use ultraviolet light.

Certain improvements are disclosed.

### BRIEF DESCRIPTION

In some illustrative embodiments disclosed herein, a disinfection light source comprises one or more disinfection light emitters (for example, LEDs configured to emit ultraviolet light: UV), and electronics configured to conduct electric power received by the electronics to the one or more disinfection light emitters to drive the one or more disinfection light sources to emit light. The electronics include a safety interlock circuit that includes: an ammeter connected to measure electric current flowing through the one or more disinfection light emitters; an analysis circuit connected to analyze the electric current measured by the ammeter; a timer circuit configured to (i) run over a delay interval while the analysis circuit detects a high irradiation condition and to (ii) stop running and reset if the analysis circuit ceases to detect the high irradiation condition; and a ‘safety’ switch connected to interrupt the conduction of the electric power to the one or more disinfection light emitters in response to the running timer circuit completing the delay interval. In some embodiments, the analysis circuit comprises a comparator connected to compare the electric current measured by the ammeter with an electrical reference value, and the timer circuit is configured to run over the delay interval while the comparator indicates the electric current measured by the ammeter exceeds the electrical reference value. If however, the comparator indicates the electrical current ceases to exceed the electrical reference value, then the timer stops running and resets. The timer may, for example, comprise a programmable digital timer having the delay interval set in hardware by circuit components including at least a resonant circuit connected with the programmable digital timer. This timer will elapse upon reaching its programmed interval if the comparator senses electric current to the light emitter continually exceeding the electrical reference value. When the timer elapses, the ‘safety’ switch is opened and current to the light emitter ceases. In some embodiments, an electronics processor providing electrical power to the disinfection light source (also referred to herein as a disinfection system controller) can operate the disinfection light source in the high irradiation condition based on information known to the electronics processor (e.g. knowledge that the environment is vacant of human occupants based on occupancy sensor readings). In this case, the disinfection system controller continues to demonstrate that it remains functional by occasionally briefly decreasing electric current below the electrical reference value (referred to herein as reset time periods), which causes the timer circuit to stop running and reset. So long as the intervals between the reset time periods are shorter than the delay interval of the timer circuit, the disinfection light source will continue running at the high irradiation condition (except for brief reductions in irradiation during the reset time periods).

Optionally, another level of ‘safety’ check is provided by the safety interlock circuit being configured to immediately operate the safety switch to interrupt the conduction of the electric power to the one or more disinfection light emitters in response to the comparator indicating the electric current measured by the ammeter exceeds an immediate shutoff electrical reference value that is higher than the electrical reference value that causes the timer circuit to run over the delay interval. This additional level compares the electrical current to the light source versus a fixed level (‘excess current’) that exceeds any foreseen level to be applied by the electronic processor mentioned above, even during vacancy of the environment. When the immediate shutoff electrical reference value is exceeded, the ‘safety’ switch is immediately opened (i.e. supersedes the timer circuit), thereby immediately interrupting current to the light-emitter.

Optionally, another level of ‘safety’ check is provided by a sub-circuit of the safety interlock circuit that measures the electrical power-supply to the safety interlock circuit itself. Unless and until this power-supply level is attained (i.e. indicating that the other safety checks are functional), the ‘safety’ switch is immediately opened, interrupting current to the light emitter.

In some illustrative embodiments disclosed herein, a disinfection system includes a disinfection light source as set forth in the immediately preceding paragraph, and a disinfection system controller comprising a microprocessor programmed to control a power supply to deliver the electric power that is received by the electronics via an associated electrical cable connecting the disinfection system controller and the disinfection light source. The disinfection system controller is not part of the unitary light source and is not disposed on or in the circuit board of the disinfection light source.

In some illustrative embodiments disclosed herein, a disinfection system includes a disinfection light source as set forth above, and a disinfection system controller connected to deliver the electric power that is received by the electronics via an associated electrical cable connecting the disinfection system controller and the disinfection light source. The disinfection system controller in these embodiments comprises a power supply and a microprocessor programmed to control the power supply to deliver the excess electrical power interspersed with reset time periods of reduced or zero electrical power wherein the interspersed reset time periods occur at intervals shorter than the delay interval of the timer circuit. The excess electrical power is effective for the analysis circuit to detect the high irradiation condition, and the reduced or zero electrical power is effective for the analysis circuit to not detect the high irradiation condition during the reset time periods.

In some illustrative embodiments disclosed herein, a disinfection light source comprises one or more disinfection light emitters comprising LEDs configured to emit ultraviolet light, and electronics configured to conduct electric power received by the electronics to the one or more disinfection light emitters to drive the one or more disinfection light sources to emit ultraviolet light. The electronics include a safety interlock circuit that includes: an ammeter connected to measure electric current flowing through the one or more disinfection light emitters; a comparator connected to compare the electric current measured by the ammeter with an electrical reference value; a timer circuit configured to run over a delay interval while the comparator indicates the electric current measured by the ammeter exceeds the electrical reference value and to stop running and reset if the comparator ceases to indicate the electric current measured by the ammeter exceeds the electrical reference value; and a switch connected to interrupt the conduction of the electric power to the one or more disinfection light emitters in response to the running timer circuit completing the delay interval.

In some illustrative embodiments disclosed herein, a disinfection method operates in conjunction with a disinfection light source as set forth in the immediately preceding paragraph. The disinfection method comprises: delivering electrical power to the disinfection light source at an electrical power that is effective for the comparator to indicate the electric current measured by the ammeter exceeds the electrical reference value; and while delivering the electrical power, delivering a reduced electrical power during reset time periods wherein the reset time periods repeat at intervals shorter than the delay interval of the timer circuit. The reduced electrical power is effective for the comparator to not indicate the electric current measured by the ammeter exceeds the electrical reference value during the reset time periods.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 diagrammatically illustrates a disinfection system configured to disinfect an environment for human occupancy (top portion), and an electrical schematic of the light source of the disinfection system (bottom portion).

FIG. 2 diagrammatically illustrates a suitable implementation of the timer of FIG. 1 as a programmable digital timer having a delay interval set in hardware by circuit components including a resonant circuit connected with the programmable digital timer and a DIP switch bank connected to set the counter period of the programmable digital timer.

FIG. 3 diagrammatically illustrates a control method suitably performed by the disinfection system controller of the disinfection system of FIG. 1.

FIG. 4 diagrammatically illustrates a disinfection light source powered by a D.C. voltage supplied by a battery (top portion), and an electrical schematic of the light source (bottom portion).

### DETAILED DESCRIPTION

“Ultraviolet (UV) radiation” or “UV light” pertains to the range between 100 nm and 400 nm, commonly subdivided into UVA, from 320 nm to 400 nm; UVB, from 280 nm to 320 nm; and UVC, from 100 nm to 280 nm. The violet range of light is 380-450 nm. It will be appreciated that as used herein the term “light” is intended to encompass light in the visible light range (typically considered 400-700 nm) and also UV light, as well as near infrared light (up to about 3000 nm).

There is a potential hazard with the emission of some UV wavelengths. To address this, there are published regulatory limits for permitted dose exposure of humans to UV light, referred to as the actinic dose Limit. The Actinic UV hazard exposure limit for exposure to ultraviolet radiation incident upon the unprotected skin or eye applies to exposure within a specified time period, which is typically any 24-hour period, or possibly any 8-hour period. To protect against injury of the eye or skin from ultraviolet radiation exposure produced by a broadband source, the effective integrated spectral irradiance (effective radiant exposure, or effective dose), Eₛ, of the light source shall not exceed 30 J/m². The effective integrated spectral irradiance, Eₛ, is then defined as the quantity obtained by weighting spectrally the dose (radiant exposure) according to the actinic action spectrum value at the corresponding wavelength. One suitable actinic action spectrum is the published IESNA Germicidal action spectrum. Electronics disclosed herein are intended to prevent foreseeable faults in the disinfection system from reaching these dose limits and thus creating a potential hazard.

U.S. Pub. No. 2016/0271281 A1 discloses disinfection systems that includes a light source configured to generate ultraviolet light toward one or more surfaces or materials in an environment for human occupancy (e.g. a room in a house or building, sometimes referred to herein for brevity as an “occupied space” although it may or may not actually be occupied at any given time) to inactivate one or more pathogens on the one or more surfaces or materials. As disclosed therein, ultraviolet light within or partly encompassing the UVA range (e.g. 280-380 nm inclusive, or in other embodiments 300-380 nm inclusive) is particularly effective for inactivating pathogens, especially bacterial pathogens. Without being limited to any particular theory of operation, it is believed that UVA is typically efficacious in inactivating bacteria by depositing its energy in the outer membrane of the cell, or the cell wall, where the energy of the UVA photon is sufficient to create reactive oxygen species (ROS) or to drive other chemical reactions that may cause enough damage to the cell envelope to kill or inactivate the bacterium.

Light at other wavelengths can also be effective for inactivating pathogens of various types and/or in various environments (e.g. bare airborne pathogen, airborne pathogen within breath aerosols, surface-bound pathogens). For example, ultraviolet light in the UVC range can be particularly effective for inactivating viral pathogens. Disinfection systems employing ultraviolet light at multiple wavelengths and/or multiple wavelength ranges is also contemplated, e.g. a combination of UVA and UVC light emitters which can provide inactivation of a range of pathogens of different types (e.g., bacterial, viral, and/or fungal, or various subgroups of these broad pathogen classifications).

In general, the higher the intensity of UV light, the more effectively the pathogen is inactivated. However, in the case of an occupied (or possibly occupied) space, the intensity of UV light is limited by an upper limit on ultraviolet dose imposed by the Near UV or Actinic UV hazard exposure limits. Hence, it is desirable to operate the light source at close to, but below, the hazard exposure limit. Notably, the hazard exposure limit is a dose value, that is, a time-integrated value. Accordingly, the designed UV intensity of a light source intended for use in disinfecting an environment for human occupancy is suitably chosen on the basis of factors including the relevant hazard exposure limit (e.g., the actinic limit for a UV light source), the spatial separation between the light source and any human occupants of the environment, and the maximum credible time period an occupant may occupy the environment. The actinic limit is wavelength-dependent. The design-basis time period is dependent upon occupational patterns of the environment. For example, in an office, an 8-hour time period may be appropriate, corresponding to a typical work shift. If some workers may put in overtime, then the design-basis time period may need to be extended to, for example, a time period of 16-hours to accommodate the possibility of a worker performing a double-shift. In some regulatory schemes, the design-basis time period is 24 hours, that is, the regulations limit the total actinic dose that may be delivered in any 24 hour period.

Regarding spatial separation between the light source and any human occupants, this is a function of the geometry of the environment and the placement of the light source in that environment. For example, in an office with 2.5-meter high ceilings, and assuming occupants standing upright with head-level being about 2.1 meters above the floor, the design-basis separation may be 0.4 meters. On the other hand, a light source intended for use in the confined space of an ambulance may have a much smaller design-basis separation, perhaps only a few centimeters.

One way to enhance efficacy of an ultraviolet disinfection system operating in an environment for human occupation is to include sensor-based logic to adjust the UV light intensity on the basis of actual occupancy as measured by passive infrared (PIR) occupancy sensors, motion sensors, or the like. In such a system, the UV intensity can be increased to a value that, if time-integrated over the design-basis time interval, results in a dose that is above the actinic hazard exposure limit whenever the environment is detected to be unoccupied. Even more finely tuned control is contemplated, such as controlling UV intensity on the basis of individual occupants, e.g. as sensed using RFID identification tags worn by occupants.

Another example of a permissible high irradiation condition is as follows. Given the actinic UV hazard exposure limit over a design-basis time period (e.g., 24 hours for example), a maximum average irradiance output by the UV light source can be calculated that, if delivered as a constant irradiance over the design-basis time period (e.g., over 24 hours), would result in delivery of a dose that is close to, but under, the actinic UV hazard (dose) exposure limit. However, this maximum average irradiance output can be safely exceeded for a limited time interval that is below the design-basis time period, so long as the “excess” dose produced by this high irradiance condition is offset by lower irradiance in some other time interval(s) of the design-basis time period. For example, consider a situation in which the UV light source receives electrical power from a disinfection system controller. If that controller detects an event likely to produce airborne pathogens, such as a cough detected using a microphone, then the controller might increase the irradiance output by the UV light source above the calculated maximum average irradiance for some time interval (say, 15 minutes) after detecting the cough. This is permissible so long as the high irradiation over the (e.g.) 15 minute time interval is offset by some other time interval during which the irradiance output by the UV light source is below the calculated maximum average irradiance, such that the total time-integrated irradiance over the design-basis time interval remains below the actinic UV hazard exposure limit.

However, sensor-based control has some disadvantages. It substantially increases cost of the disinfection system. Since safety is implicated, such a sensor-controlled system usually will require redundant sensors, preferably of different modalities, to avoid erroneous determinations of vacancy when the environment is in fact occupied. To avoid a potential single point of failure, sensor-based control may employ a communication network with wired and/or wireless communication pathways and either a central controller (e.g. a computer) or a distributed control network (e.g. a mesh network). But the communication network itself introduces a point of possible failure, e.g. accurate control signals from the network may not reach the light source due to a network problem. Yet another point of possible failure lies in the software or firmware implementing the control. For example, a software error may lead to a light source being driven at an intensity that is higher than the design-basis intensity.

Other disinfection light sources may not employ occupancy sensors or related control. These disinfection light sources are designed to operate at a UV intensity that (for the geometry of the placement of the disinfection light source in the environment) neve exceeds the actinic limit. Such a light source preferably is configured to a preset intensity that is safe over a design-basis time period (e.g. 24 hours) at the factory, and thereafter is operated at that preset intensity. However, in such as design, a problem can arise if the preset intensity is later changed to an unsafe value; or, if failure of a component of the electronics causes the light source to output at an intensity that is above the factory-preset intensity.

In view of such concerns, disclosed herein are disinfection light sources with on-board hardware that provides protection against problems in the network-sourced control, as well that provides protection as against hardware component failures in the disinfection light source itself.

It will be appreciated that implementing reliable hardware-based protection against the disinfection light source being operated at a high irradiation condition in an uncontrolled manner due to internal hardware component failure or erroneous control signals delivered via a control network is difficult. The hardware-based protection cannot rely in any way on control information obtained via any control network, since the hardware-based protection is intended to provide a second level of security in the event of erroneous control information being received (or, in some embodiments such as a battery-driven UV light source, there may be no control network connected to the light source at all). The hardware-based protection should also be robust against internal electronic component failures, short circuits, or the like which might occur within the light source itself. At the same time, the hardware-based protection is preferably robust against minor glitches in the network control signals (if present) or supplied electric power. This is because continued operation of the disinfection UV light source is itself safety-related, insofar as deactivation of the UV light source deprives occupants of protection against the pathogens the UV light source is designed to inactivate.

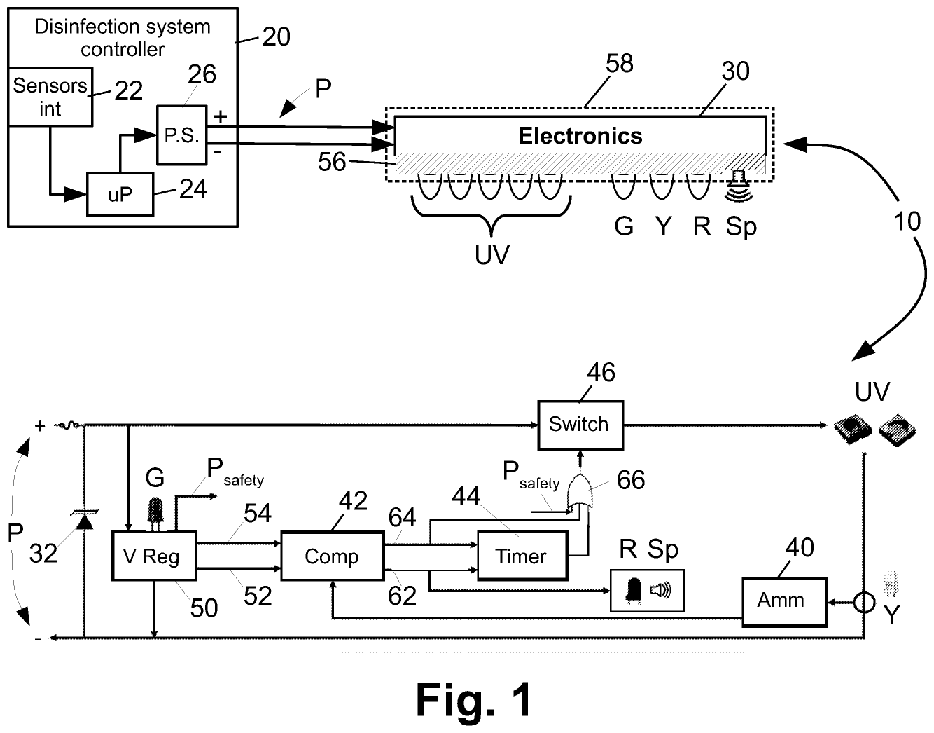

With reference to FIG. 1, an illustrative disinfection light source 10 is shown in diagrammatic view (top drawing) and as a simplified electrical schematic (bottom drawing). The light source 10 includes one or more disinfection light emitters, which in the illustrative embodiment are ultraviolet (UV) light emitting diodes (LEDs) UV. The choice of the one or more disinfection light emitters is made during design and construction of the disinfection light source 10 on the basis of the pathogens which are to be disinfected and optionally other considerations. For viral pathogens, the one or more disinfection light emitters preferably emit ultraviolet light, with UVC LEDs (that is, LEDs whose dominant emission peak is in the UVC spectral range) being particularly efficacious for viral pathogens. For bacterial pathogens, the one or more disinfection light emitters also preferably emit ultraviolet light, with UVA LEDs (that is, LEDs whose dominant emission peak is in the UVA spectral range) being particularly efficacious for bacterial pathogens. It is also contemplated that the one or more disinfection light emitters may include two, three, four, or more LEDs that emit light at different dominant emission peaks. For example, the one or more disinfection light emitters may include a first group of one or more UVC LEDs and a second group of one or more UVA LEDs, such a combination being particularly efficacious against both viral and bacterial pathogens. Moreover, it is contemplated for the one or more disinfection light emitters to include light sources other than LEDs, such as laser diode light sources, vertical cavity surface emitting lasers (VCSELs), or so forth.

The illustrative disinfection light source 10 also includes three indicator LEDs, namely a green indicator LED G, a yellow indicator LED Y, and a red indicator LED R. In general, the green indicator LED G, when lit, indicates the disinfection light source 10 is receiving electrical power (or, if powered by an internal battery, indicates the internal battery is adequately charged). The yellow indicator LED Y, when lit, indicates the disinfection light source 10 is operating to output disinfection light. (This is useful of the disinfection light output by the one or more disinfection light sources UV is not visible to the human eye, which is generally the case for UV LEDs). As an alternative to providing the yellow indicator LED Y, it is contemplated to coat a portion of the disinfection light source with a phosphorescent or fluorescent layer that absorbs UV light and converts it to visible light. If such a coating is provided and positioned to receive a portion of the UV light output by the one or more UV disinfection light sources UV, then the phosphorescence or luminescence produced by this coating would serve as the indication that the disinfection light source(s) UV are operating.

The red indicator LED R, when lit, indicates the disinfection light source 10 is operating to output disinfection light at an intensity that is higher than the design-basis intensity. Additional or alternatively, sounding of a loudspeaker Sp may serve this purpose. It is noted that illumination of the red indicator LED R (and/or sounding of the loudspeaker Sp) does not necessarily indicate an immediate hazard is present, or even that the disinfection light source 10 is operating in an unexpected operating mode. For example, in a communication network-controlled setting in which the occupancy sensors include a microphone, it is contemplated for the communication network to (over)drive the disinfection light source(s) UV for a brief period after detecting a cough or other sound indicative of an occupant (person) producing a substantial aerosolized outflow of breath. However, illumination of the red indicator LED R (and/or sounding of the loudspeaker Sp) does provide a visible (and/or audible) indication that the disinfection light source(s) UV are being overdriven.

FIG. 1 further diagrammatically illustrates a disinfection system controller 20, which represents the disinfection system controller 20 of a communication network that controls the illustrative disinfection light source 10 and (typically) other disinfection light sources on the communication network. (These other disinfection light sources on the communication network may be other instances of the illustrative disinfection light source 10, and/or may be disinfection light sources of other type(s)). The illustrative disinfection system controller 20 includes a sensor interface 22 that receives signals from one or more occupancy sensors distributed around the environment for human occupation in which the disinfection light source 10 is operating. The disinfection system controller 20 further includes a microprocessor 24 that is programmed to perform control operations, such as analyzing the signals received from the occupancy sensors at the sensors interface 22 to determine whether the environment is occupied adjusting the UV output of the disinfection light source 10 on the basis of the occupancy determination.

The disinfection system controller 20 further includes (or controls) a power supply 26 that delivers electrical power P to the disinfection light source 10. The delivered electrical power P may be a controlled voltage (in which case the power supply 26 is a voltage power supply) or a controlled electric current (in which case the power supply 26 is a current supply). The disinfection system controller 20 can control the UV light intensity output by the UV light emitter(s) UV by controlling the power supply 26 to adjust the electrical power P delivered to the disinfection light source 10.

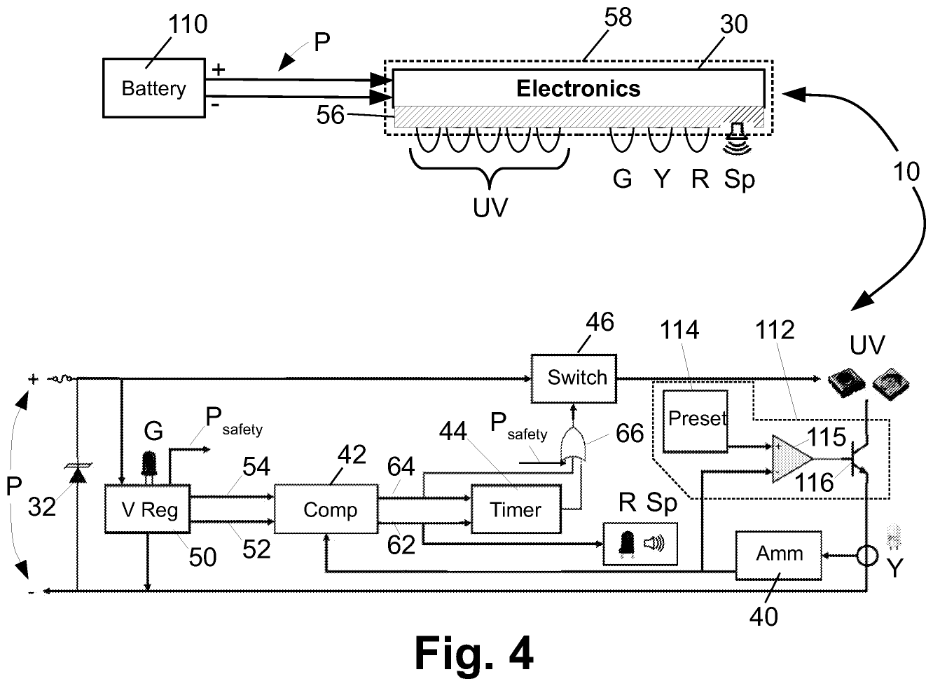

The disinfection light source 10 further includes electronics 30 configured to conduct the electric power P received by the electronics 30 to the one or more disinfection light emitters UV to drive the one or more disinfection light sources UV to emit light. As seen in the simplified electrical schematic shown in the bottom of FIG. 1, the electronics 30 include an illustrative Zener diode 32 that operates as a safety fuse. (Alternatively or additionally, a fuse, circuit breaker, or the like may be similarly provided). The electronics 30 further comprise a safety interlock circuit that includes: an ammeter 40 connected to measure electric current flowing through the one or more disinfection light emitters UV; an analysis circuit 42 connected to analyze the electric current measured by the ammeter; a timer circuit 44 configured to run while the analysis circuit 42 detects a high irradiation condition; and a switch 46 connected to interrupt the conduction of the electric power P to the one or more disinfection light emitters UV in response to the running timer circuit 44 completing a delay interval of the timer circuit 44. The analysis circuit 42 in the illustrative embodiment comprises a comparator 42 connected to compare the electric current measured by the ammeter 40 with an electrical reference value 52, which is a calculated value to ensure that irradiance of the UV light emitter(s) UV is below the actinic dose limit (and thus is at a safe level). The reference value 52 is pre-calculated (and optionally is adjustable) to account for the installed ceiling-height of the light source 10. The ceiling height of the light source 10 impacts the highest safe level of irradiance, because of the relationship of distance vs lumens coming from the inverse-square law of physics, as well the electrical-to-UV light conversion efficiency of the UV light emitter(s) UV (that is, its relation of electrical current to lumens). The optional adjustment of this reference value 52 is performed by a sub-circuit 112 including in the illustrative embodiment (see FIG. 4) at least a potentiometer 114 (which may be analog or digital), which is configured to adjust the electric current measured by the ammeter 40 based on the setting of the potentiometer 114. In the case of the comparator 42 measuring electric current as being below this reference value 52, that current is permitted to continue to flow to drive the UV light emitter(s) UV, and the ‘safety’ switch 46 is not opened to interrupt the electric current. The safety interlock circuit further includes a voltage regulator 50 that is driven by the received electrical power P and outputs: power P_{safety} that powers the remainder of the safety interlock circuit and at least one electrical reference value (in the illustrative example, a first electrical reference value 52 and a second electrical reference value 54 that is higher than the first electrical reference value 52. The second electrical reference value 54 is also referred to herein as an immediate shutoff electrical reference value. The voltage regulator 50 also drives the green indicator LED G to indicate the disinfection light source 10 is receiving electrical power.

The disinfection light source 10 may be variously physically embodied. For example, the one or more disinfection light sources UV and indicator LEDs G, Y, R and/or loudspeaker Sp may be mounted on a circuit board 56, with the electronics 30 mounted on a backside of the circuit board 56 opposite the side supporting the light sources and indicator LEDs (as shown) and/or on the same side as the side supporting the light sources and indicator LEDs (not shown). Hence, in this approach the electronics 30 and the one or more disinfection light emitters UV (and the indicators G, Y, R, Sp) are mounted on the circuit board 56 to form the disinfection light source 10 as a unitary light source. (Note that the disinfection system controller 20 is not part of the unitary light source and is not mounted on the circuit board 56 of the disinfection light source 10). Optionally, a protective and/or cosmetic housing 58 (diagrammatically indicated by a dashed line) may be provided for the disinfection light source 10, with openings through which the UV and indicator light passes. These are merely illustrative physical configurations, and the disinfection light source 10 can be constructed with other form factors, shapes, and/or so forth.

The illustrative analysis circuit 42 comprises a comparator 42 connected to compare the electric current measured by the ammeter 40 with the (first) electrical reference value 52 to output an indicator signal 62 indicating whether the electric current measured by the ammeter 40 exceeds the (first) electrical reference value 52. The timer circuit 44 is configured to run over a delay interval while the comparator 42 indicates the electric current measured by the ammeter 40 exceeds the (first) electrical reference value 62; and to stop running and reset if the comparator 42 ceases to indicate the electric current measured by the ammeter 40 exceeds the electrical reference value 62. As shown in the electrical schematic at the bottom of FIG. 1, the indicator signal 62 indicating whether the electric current measured by the ammeter 40 exceeds the (first) electrical reference value 52 is also used to drive the red indicator LED R and/or the loudspeaker Sp to indicate the disinfection light source 10 is operating to output disinfection light at an intensity that is higher than the design-basis intensity (which corresponds to the first electrical reference value 52). That is, the red indicator LED R and/or loudspeaker Sp indicates the disinfection light source 10 is operating at a high irradiation condition. This high irradiation condition is not necessarily an unsafe condition. As explained previously, the disinfection system controller 20 may operate the disinfection light source 10 in a high irradiation condition if, for example, sensor data received at the sensors interface 22 indicates the environment is unoccupied. As another example, the disinfection system controller 20 may operate the disinfection light source 10 in a high irradiation condition for a brief time interval if, for example, a cough or other event indicative of a possible airborne pathogen release is detected, so long as the controller 20 compensates for the excess dose delivered during this brief time interval by some other time interval in which the disinfection light source 10 is operated at some lower irradiation level (such that the total dose over the design-basis time period, e.g. 24 hours under some regulatory schemes, is below the actinic hazard exposure limit).

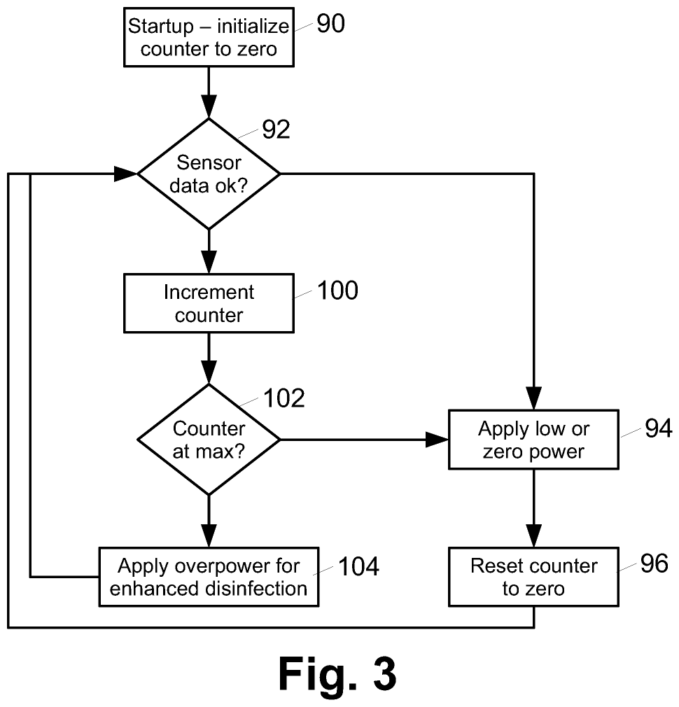

The first electrical reference value 52 is chosen to serve as surrogate for directly measuring the irradiance output by the UV LEDs. Thus, when the electric current measured by the ammeter 40 exceeds the (first) electrical reference value 52, this indicates that the UV LEDs are outputting an irradiance that is above the level posing a potential hazard to a human if it is continued indefinitely (or at least if it is continued over the design-basis time period). This again does not necessarily indicate a malfunction, as an upstream control system such as the illustrative disinfection system controller 20 may be intentionally demanding that irradiance, e.g. while the sensor readings acquired at the sensors interface 22 indicate that humans are absent from the environment, or for some brief time interval following a detected cough. As will be discussed later herein with reference to FIG. 3, the use of the timer circuit 44 to delay switch-off of the electrical current to the UV LEDs advantageously allows for such upstream control system to drive the UV LEDs above the level posing a potential hazard to a human, so long as the upstream control system provides ongoing indications that it is actively controlling to drive the UV LEDs at this high level.

This portion of the illustrative safety interlock circuit controlled by the indicator signal 62 indicating whether the electric current measured by the ammeter 40 exceeds the (first) electrical reference value 52 operates on a time delay principle, and advantageously allows the electric current measured by the ammeter 40 to exceed the design-basis intensity for a time interval defined by the delay interval of the timer circuit 44 before the safety interlock circuit triggers the switch 46 to interrupt the conduction of the electric power P to the one or more disinfection light emitters UV (and thereby stop the UV emission). Thus, for example, if the disinfection system controller 20 determines, based on occupancy sensor data collected by the sensors interface 22, that the disinfection light emitter(s) UV should be run above the design-basis intensity for a brief time (e.g., due to a cough event detected by a microphone) then this can be done. However, a disadvantage of this time delay-based safety interlock is that if the UV intensity is too high then this brief period of permitted operation can potentially present an actinic hazard to occupants of the environment in which the disinfection light source 10 operates.

To address this problem, in the illustrative safety interlock circuit the comparator 42 also compares the electric current measured by the ammeter 40 with the second electrical reference value 54, which is also referred to herein as the immediate shutoff electrical reference value. The safety interlock circuit is configured to immediately operate the switch 46 to interrupt the conduction of the electric power P to the one or more disinfection light emitters UV in response to the comparator 42 indicating (by way of a second indicator signal 64) that the electric current measured by the ammeter 40 exceeds the immediate shutoff electrical reference value 54 (which, again, is higher than the electrical reference value 52 used in the time delay-based safety interlock). In the electrical schematic shown at the bottom of FIG. 1, this dual operation is enabled by way of a logical “or” gate 66 that combines the output of the timer circuit 44 and the second indicator signal 64 as a logical “or”. That is, the output of the logical “or” gate 66 is logical “one” if at least one of the following is true: (1) the running timer circuit 44 completes its delay interval, or (2) the second indicator signal 64 indicates that the electric current measured by the ammeter 40 exceeds the immediate shutoff electrical reference value 54. The immediate shutoff electrical reference value 54 is set to correspond to a UV intensity output of the disinfection light emitter(s) exceeding some pre-selected unsafe condition (or, alternatively exceeding some level that is not credibly expected to be demanded by the disinfection system controller 20).

A further potential hazard condition could arise if the safety interlock circuit itself malfunctions. A likely scenario in which this could happen is of the voltage regulator 50 or other source of electrical power for the safety interlock circuit malfunctions such that operational electrical power to the safety interlock circuit falls below a minimum operational power level for reliable operation of the safety interlock circuit. To address this potential hazard condition in the illustrative example of FIG. 1, the logical “or” gate 66 is a wired or gate. In this design, the passive transistor shutoff connected to operate the switch 46 to interrupt the conduction of the electric power to the disinfection light emitter(s) UV in response to the operational electrical power (denoted P_{safety} in FIG. 1) to the safety interlock circuit being below the minimum operational power level. This ensures that if the minimum operational power level is not provided (potentially thereby resulting in unreliable operation of the ammeter 40, comparator 42, and/or timer 44) then the switch 46 will be opened to turn off the disinfection light emitter(s) UV.

Hence, the switch 46 is connected to interrupt the conduction of the electric power to the one or more disinfection light emitters UV in response to any of: (i) a malfunction indicated by the ammeter (e.g. light emitter over-current, as indicated by the electric current measured by the ammeter 40 exceeding the immediate shutoff electrical reference value 54), or (ii) the timer circuit 44 running and completing its delay interval thereby indicating the failure of an upstream controller (e.g. the disinfection system controller 20) in performing dosing calculations, or (iii) the level of power to the safety interlock circuit (that is, the power P_{safety} indicated in FIG. 1) being insufficient to guarantee proper operation of the safety interlock circuit.

In FIG. 1, the illustrated comparator 42 compares the electric current measured by the ammeter 40 with both the (first) reference value 52 controlling the time delay-based safety interlock and with the immediate shutoff electrical reference value 54, so as to generate the (first) indicator signal 62 and the (second) indicator signal 64, respectively. Although not shown, it will be appreciated that the illustrative comparator 42 can be implemented as two comparators: a first comparator that compares the electric current measured by the ammeter 40 with the (first) reference value 52 and outputs the (first) indicator signal 62 indicating whether the electric current measured by the ammeter 40 exceeds the (first) electrical reference value 52 controlling the time delay-based safety interlock; and a second comparator that compares the electric current measured by the ammeter 40 with the immediate shutoff electrical reference value 54 and outputs the (second) indicator signal 64 indicating whether the electric current measured by the ammeter 40 exceeds the immediate shutoff electrical reference value 54.

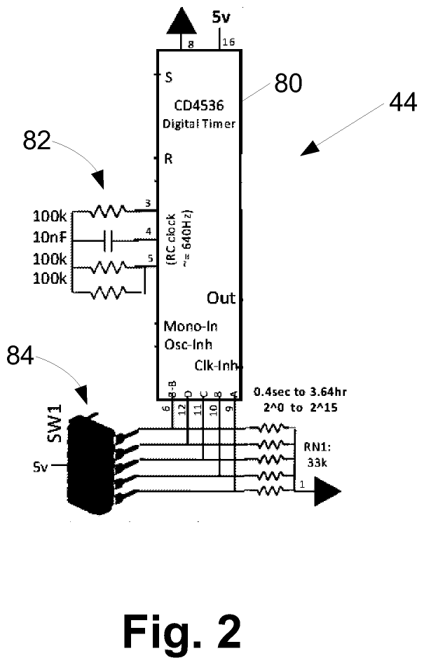

With brief reference to FIG. 2, one suitable embodiment of the timer circuit 44 comprises a CD4536 CMOS programmable timer integrated circuit (IC) 80. A suitable configuration of the CD4536 80 for setting the delay interval includes an RC resonant circuit 82 connected to form a local RC clock for the CD4536 IC 80, and a DIP switch bank 84 for setting the counter period (inputs A, B, C, and D of the CD4536 IC 80) in hardware. The combination of the resonance frequency of the RC resonant circuit 82 and the counter period set by the DIP switch bank 84 defines the delay interval of the thusly configured CD4536 IC timer.

As previously noted, the delay interval-based safety interlock implemented by the portion of the illustrative safety interlock circuit controlled by the indicator signal 62 indicating whether the electric current measured by the ammeter 40 exceeds the (first) electrical reference value 52 operates on a delay interval principle, and advantageously allows the electric current measured by the ammeter 40 to exceed the design-basis intensity for the delay interval of the timer circuit 44 before the safety interlock circuit triggers the switch 46 to interrupt the conduction of the electric power P to the one or more disinfection light emitters UV (and thereby stop the UV emission). Thus, for example, if the disinfection system controller 20 determines, based on occupancy sensor data collected by the sensors interface 22, that the disinfection light emitter(s) UV should be run above the design-basis intensity for a brief time (e.g., due to a cough event detected by a microphone) then this can be done. However, a disadvantage of this delay interval-based safety interlock is that if the UV intensity is too high then this brief period of permitted operation can potentially present an actinic hazard to occupants of the environment in which the disinfection light source 10 operates

In the case of FIG. 1 in which the disinfection system controller 20 is controlling the disinfection light source 10, it may be desirable to allow for the controller 20 to cause the disinfection light source 10 to output UV light that exceeds the design-basis intensity for a time period that is longer than the delay interval of the timer circuit 44. For example, if the controller 20 determines the environment in which the disinfection light source 10 is operating is unoccupied for an extended time period (e.g., tens of minutes or even an hour or more) then it may be useful to cause the disinfection light source 10 to output UV light that exceeds the design-basis intensity during that period of unoccupancy to provide enhanced disinfection of pathogens.

On the other hand, when the disinfection system controller 20 causes the disinfection light source 10 to output UV light that exceeds the design-basis intensity for an extended time period, it would be advantageous for the safety interlock circuit of the disinfection light source to operate to ensure that the disinfection system controller 20 is actually providing positive control to exceed the design-basis intensity for this extended time period. This is because there could be situations in which the disinfection system controller 20 may fail to provide positive control. For example, if the disinfection system controller 20 could experience an internal failure such as the power supply 26 malfunctioning and outputting an excessively high power P; or, a software or firmware error could cause the microprocessor 24 to freeze up and cease to control operation of the power supply 26. These are merely examples of possible failure modes. In such cases, the power supply 26 may be outputting a high level of power P causing the disinfection light source 10 to output UV light that exceeds the design-basis intensity, but not under positive control of the controller 20.

Advantageously, the disclosed safety interlock circuit can operate to ensure that the disinfection system controller 20 is actually providing positive control when exceeding the design-basis intensity for an extended time period. This can be done in conjunction with suitable programming of the microprocessor 24 of the disinfection system controller, as described next.

With continuing reference to FIGS. 1 and 2 and with further reference to FIG. 3, an example of such cooperative operation of the disinfection system controller 20 and the safety interlock circuit of the disinfection light source 10 is described. In this example, it is assumed that the delay interval of the timer circuit 44 is set to a relatively short value, e.g. the delay interval may be one second, or a few seconds, or ten seconds, or so forth. As previously described with reference to FIG. 2, the delay interval is set configuring the RC resonant circuit 82 to a resonance frequency and the DIP switch bank 84 to a counter period so as to define the desired delay interval of the non-limiting illustrative CD4536 IC timer 80. On the other hand, it is assumed for this example that the disinfection system controller 20 is to cause the disinfection light source 10 to output UV light that exceeds the design-basis intensity for a time period that is longer than the delay interval of the timer circuit 44. For example, the disinfection system controller 20 may cause the disinfection light source 10 to output UV light that exceeds the design-basis intensity for a time period of two minutes, or ten minutes, or an hour, or longer. To do so, in brief, the disinfection system controller 20 is programmed to deliver the electric power P as excess electrical power with interspersed reset time periods of reduced or zero electrical power. The interspersed reset time periods occur at intervals shorter than the delay interval of the timer circuit 44. The excess electrical power is effective for the analysis circuit (e.g. comparator 42) to detect the high irradiation condition (e.g., UV output exceeding the design-basis intensity). The reduced or zero electrical power is effective for the analysis circuit to not detect the high irradiation condition during the reset time periods.

With reference to FIG. 3, a suitable implementation of programming of the microprocessor 24 of the disinfection system controller 20 to provide excess electrical power with interspersed reset time periods of reduced or zero electrical power starts by initializing a counter to zero in an operation 90. In an operation 92, the microprocessor 24 analyzes the sensor data acquired by the sensors interface 22 to verify that it is safe to start/continue to deliver excess electrical power. For example, the operation 92 may verify that occupancy sensors continue to provide valid data which indicates that the environment is unoccupied. If at any time the operation 92 detects that it is not safe to continue to deliver excess electrical power (for example, because occupancy is detected, or because the occupancy sensor data is not valid, e.g. a reading of zero volts indicating loss of connectivity to the sensor) then flow passes to an operation 94 in which the power P is reduced to a level that effective to operate the disinfection light source 10 at or below its design-basis UV intensity, and in an operation 96 the counter is reset to zero.

On the other hand, if the operation 92 detects that it is safe to continue to deliver excess electrical power to the disinfection light source 10, then flow passes to an operation 100 at which the counter is incremented. At a decision 102, it is determined whether the counter is at a maximum value. Assuming the maximum counter value has not yet been reached, at an operation 104 the microprocessor 24 of the controller 20 controls the power supply 26 to start or continue to deliver the electrical power P at the excess electrical power level effective to drive the disinfection light source 10 to output UV light at above its design-basis intensity level. Flow then passes back to operation 92 to iteratively perform the loop of operations 92, 100, 102, 104 until at the decision operation 102 it is detected that the counter has reached the maximum value. This maximum counter value is set so that the time for counting from zero (as set in operation to the maximum value is less than the delay interval of the timer 44 of the safety interlock circuit of the disinfection light source 10. When the decision 102 detects the counter has reached maximum, then flow passes to operation 94 to implement a reset time period of reduced or zero electrical power via the operation 94. The counter is then reset to zero at operation 96 and flow passes back to the loop of operations 92, 100, 102, 104 to continue delivering the excess electrical power.

It will be appreciated that if the reset time periods occur at time intervals (controlled by the maximum counter value implemented at decision operation 102) that are shorter than the delay interval of the timer circuit 44, then the effect of the reset time periods will be to stop and reset the running timer circuit 44 before it completes its delay interval. Hence, the switch 46 will never interrupt the conduction of the electric power to the disinfection light emitter(s) UV in response to the running timer circuit completing its delay interval. With the exception of brief intervals of reduced UV light output corresponding to the reset time periods, the controller 20 executing the loop of operations 92, 100, 102, 104 will be able to drive the disinfection light source 10 with excess electrical power (that is, at above its design-basis UV light intensity output level) indefinitely.

On the other hand, if the disinfection system controller 20 experiences a malfunction such as the power supply 26 malfunctioning and outputting an excessively high power P, or a software or firmware error causing the microprocessor 24 to freeze up and cease to control operation of the power supply 26, then the effect is that the power supply 26 will output the excess electrical power continuously, without interspersed reset time periods of reduced or zero electrical power. As a consequence, the timer circuit 44 will not be reset by the operation 94 and in the presence of such a malfunction of the controller 20 the switch 46 will interrupt the conduction of the (excess) electric power P to the disinfection light emitter(s) UV when the running timer circuit 44 completes its delay interval. In this way, the safety interlock circuit of the disinfection light source 10 independently verifies continued positive control of the disinfection light source 10—and, if such positive control is detected to be lost (by cessation of the reset time periods), then the safety interlock circuit operates to stop the UV light emission.

In the disinfection light source 10 of FIG. 1, there is no way to set the design-basis UV light output locally on the disinfection light source 10. Rather, the design-basis UV light output is effectively set by the (first) electrical reference value 52. This may be a suitable approach in the system-level context of FIG. 1 since the disinfection system controller 20 is capable of driving the disinfection light source 10 above the design-basis UV light output as described with reference to FIGS. 1-3. However, in some implementations there may be no disinfection system controller 20, and in these settings (as well as settings that employ the controller 20), it may be desirable to provide a way for the installer to adjust the design-basis UV light output locally at the disinfection light source 10.

With reference to FIG. 4, an illustrative example of an approach for providing local adjustment of the design-basis UV light output is shown. In the illustrative example of FIG. 4, the disinfection light source 10 is driven by a battery 110 that (in the absence of some malfunction of the battery 110 or connecting electrical wiring) drives the disinfection light source 10 at a fixed power P. As seen in the simplified electrical schematic shown in the bottom of FIG. 4, the electronics 30 are similar to those of the embodiment of FIG. 1, with the exception of the addition of a design-basis UV output level adjustment sub-circuit 112. This sub-circuit 112 includes potentiometer 114 via which a user can adjust (i.e. preset) the design-basis UV intensity level. The potentiometer 114 may, for example, be a variable resistor, a rheostat, or the like that presents a user-adjustable resistance. For example, the potentiometer 114 may include a rotary control accessible through an opening in the housing 58 and adjustable using a hex screwdriver, flat screwdriver, Phillips screwdriver, or the like. As seen in FIG. 4, the design-basis UV output level adjustment sub-circuit 112 further includes an operational amplifier 115 and a transistor 116 connected to adjust the current read by the ammeter 40. Hence, the adjustment sub-circuit 112 is configured to adjust the electric current measured by the ammeter 40 based on a setting of the potentiometer 114.

As previously explained with reference to FIG. 1, the timer circuit 44 runs while the comparator 42 indicates the electric current measured by the ammeter 42 exceeds the electrical reference value, and the switch 46 interrupts the electric power to the disinfection light emitter(s) UV in response to the running timer circuit 44 completing its delay interval. As the adjustment sub-circuit 112 in the embodiment of FIG. 4 adjusts the current that is measured by the ammeter 40 (and, indeed, that same adjusted current flows through the disinfection light emitter(s) UV), the potentiometer 114 thus is operable to adjust the design-basis UV output. This allows for the user to adjust the design-basis UV output of the disinfection light source 10 of FIG. 4 to, for example, accommodate a specific ceiling height. This can be useful if, for example, the disinfection light source 10 may be installed in a room (e.g. with ceiling height on the order of 2 meters) or in an ambulance that may have a much smaller design-basis separation, perhaps only a few centimeters. Advantageously, the safety interlock circuit of the embodiment of FIG. 4 is (with the exception of the adjustment sub-circuit 112) identical with that of the embodiment of FIG. 1, and continues to operate to protect against malfunctions. The safety interlock circuit of the embodiment of FIG. 4 also operates to protect against any possibility that the user might set the potentiometer 114 too high, so that the adjusted electrical current exceeds the first electrical reference value 52. If the user adjusts the potentiometer 114 too high then the timer will start running, the red indicator light R and/or loudspeaker Sp will activate, and the user will have the delay interval to adjust the potentiometer to lower the current back to a safe level. If this is not done, then at the expiration of the delay interval the switch 46 will operate to turn off the ultraviolet light emission. (In other contemplated embodiments, the potentiometer 114 is designed so that its adjustment limits, absent some malfunction, do not allow for the adjusted electrical current to exceed the first electrical reference value 52).

In a preferred practical implementation, the potentiometer 114 is adjusted at the factory that manufactures the disinfection light source 10 of FIG. 4 to provide an initial factory setting. If/when the disinfection light source 10 is re-purposed (e.g. removed and installed at a different ceiling-height), a qualified service person (with the ‘security key’ to this setting) may adjust this factory setting. To provide further security, rather than the potentiometer 114 being adjustable using a standard hex, flat, or Phillips screwdriver, the potentiometer 114 may be adjustable using a special tool. In another implementation, the potentiometer 114 may be an electronic component that is set electronically using a secure near field communication (NFC) protocol or the like.

The present disclosure has been described with reference to exemplary embodiments. Modifications and alterations may occur to others upon reading and understanding the preceding detailed description. It is intended that the present disclosure be construed as including all such modifications and alterations insofar as they come within the scope of the appended claims or the equivalents thereof.

1. A disinfection light source comprising:
one or more disinfection light emitters; and
electronics configured to conduct electric power received by the electronics to the one or more disinfection light emitters to drive the one or more disinfection light sources to emit light, the electronics including a safety interlock circuit that includes:
an ammeter connected to measure electric current flowing through the one or more disinfection light emitters;
an analysis circuit connected to analyze the electric current measured by the ammeter;
a timer circuit configured to (i) run over a delay interval while the analysis circuit detects a high irradiation condition and to (ii) stop running and reset if the analysis circuit ceases to detect the high irradiation condition; and
a switch connected to interrupt the conduction of the electric power to the one or more disinfection light emitters in response to the running timer circuit completing the delay interval.

2. The disinfection light source of claim 1 wherein:
the analysis circuit comprises a comparator connected to compare the electric current measured by the ammeter with an electrical reference value; and
the timer circuit is configured to (i) run over the delay interval while the comparator indicates the electric current measured by the ammeter exceeds the electrical reference value and to (ii) stop running and reset if the comparator indicates the electric current measured by the ammeter ceases to exceed the electrical reference value.

3. The disinfection light source of claim 2 wherein the safety interlock circuit further includes:
a voltage regulator driven by the electric power and generating the electrical reference value.

4. The disinfection light source of claim 3 wherein the voltage regulator further extracts operational power for the safety interlock circuit from the electric power.

5. The disinfection light source of claim 2 wherein the safety interlock circuit is configured to immediately operate the switch to interrupt the conduction of the electric power to the one or more disinfection light emitters in response to the comparator indicating the electric current measured by the ammeter exceeds an immediate shutoff electrical reference value that is higher than the electrical reference value.

6. The disinfection light source of claim 1 wherein the safety interlock circuit further includes:
an adjustment sub-circuit including at least a potentiometer, wherein the adjustment sub-circuit is configured to adjust the electric current measured by the ammeter based on a setting of the potentiometer.

7. The disinfection light source of claim 1 wherein the timer circuit comprises a programmable digital timer having the delay interval set in hardware by circuit components including at least a resonant circuit connected with the programmable digital timer.

8. The disinfection light source of claim 1 further comprising:
a high irradiation condition indicator connected to produce a human-perceptible high irradiation indication in response to the analysis circuit detecting the high irradiation condition.

9. The disinfection light source of claim 1 further comprising:
an indicator LED configured to emit visible light that is connected in series with the one or more disinfection light emitters;
whereby the electric power conducted to the one or more disinfection light emitters also drives the indicator LED to emit the visible light.

10. The disinfection light source of claim 1 wherein the safety interlock circuit further includes:
a passive transistor shutoff connected to operate the switch to interrupt the conduction of the electric power to the one or more disinfection light emitters in response to operational electrical power to the safety interlock circuit being below a minimum operational power level.

11. The disinfection light source of claim 1 wherein the one or more disinfection light emitters comprise LEDs configured to emit ultraviolet light.

12. The disinfection light source of claim 1 wherein the one or more disinfection light emitters comprise LEDs configured to emit ultraviolet light having a maximum peak in a wavelength range of 240 nanometers to 280 nanometers inclusive.

13. The disinfection light source of claim 1 further comprising:
a circuit board;
wherein the electronics and the one or more disinfection light emitters are mounted on the circuit board to form the disinfection light source as a unitary light source.

14. A disinfection system comprising:
a disinfection light source as set forth in claim 13; and
a disinfection system controller comprising a microprocessor programmed to control a power supply to deliver the electric power that is received by the electronics via an associated electrical cable connecting the disinfection system controller and the disinfection light source;
wherein the disinfection system controller is not part of the unitary light source and is not disposed on the circuit board of the disinfection light source.

15. A disinfection system comprising:
a disinfection light source as set forth in claim 1; and
a disinfection system controller connected to deliver the electric power that is received by the electronics via an associated electrical cable connecting the disinfection system controller and the disinfection light source;
wherein the disinfection system controller comprises a power supply and a microprocessor programmed to control the power supply to deliver excess electrical power with interspersed reset time periods of reduced or zero electrical power wherein the interspersed reset time periods occur at intervals shorter than the delay interval of the timer circuit;
wherein the excess electrical power is effective for the analysis circuit to detect the high irradiation condition; and
wherein the reduced or zero electrical power is effective for the analysis circuit to not detect the high irradiation condition during the reset time periods.

16. The disinfection system of claim 15 further comprising:
an occupancy sensor;
wherein the disinfection system controller is connected to read the occupancy sensor and to deliver the excess electrical power during at least a portion of an unoccupied time interval in which the occupancy sensor does not detect occupancy.

17. A disinfection light source comprising:
one or more disinfection light emitters comprising LEDs configured to emit ultraviolet light; and
electronics configured to conduct electric power received by the electronics to the one or more disinfection light emitters to drive the one or more disinfection light sources to emit ultraviolet light, the electronics including a safety interlock circuit that includes:
an ammeter connected to measure electric current flowing through the one or more disinfection light emitters;
a comparator connected to compare the electric current measured by the ammeter with an electrical reference value;
a timer circuit configured to run over a delay interval while the comparator indicates the electric current measured by the ammeter exceeds the electrical reference value and to stop running and reset if the comparator ceases to indicate the electric current measured by the ammeter exceeds the electrical reference value; and
a switch connected to interrupt the conduction of the electric power to the one or more disinfection light emitters in response to the running timer circuit completing the delay interval.

18-20. (canceled)

21. The disinfection light source of claim 17 wherein the safety interlock circuit further includes:
a passive transistor shutoff connected to operate the switch to interrupt the conduction of the electric power to the one or more disinfection light emitters in response to operational electrical power to the safety interlock circuit being below a minimum operational power level.

22. The disinfection light source of claim 17 wherein the timer circuit comprises a programmable digital timer having the delay interval set in hardware by circuit components including at least a resonant circuit connected with the programmable digital timer.

23-25. (canceled)

26. A disinfection method operating in conjunction with a disinfection light source as set forth in claim 17, the disinfection method comprising:
delivering electrical power to the disinfection light source at an electrical power that is effective for the comparator to indicate the electric current measured by the ammeter exceeds the electrical reference value; and
while delivering the electrical power, delivering a reduced electrical power during reset time periods wherein the reset time periods repeat at intervals shorter than the delay interval of the timer circuit;
wherein the reduced electrical power is effective for the comparator to not indicate the electric current measured by the ammeter exceeds the electrical reference value during the reset time periods.
